(19) United States
(12) Patent Application Publication
(10) Pub. No.: US 2026/0325783 A1
(43) Pub. Date: Oct. 22, 2026

(54) DEVICE FOR DISPENSING LIQUID OR SEMI-SOLID PRODUCTS, PARTICULARLY MEDICINES, MEDICAL DEVICES OR COSMETIC PRODUCTS

(71) Applicant: IBSA INSTITUT BIOCHIMIQUE SA, Lugano (CH)

(72) Inventor: Davide MANZONI, Lugano (CH)

(21) Appl. No.: 19/483,553

(22) PCT Filed: May 10, 2024
(86) PCT No.: PCT/IB2024/054554

§ 371 Date: Nov. 11, 2025

(30) Foreign Application Priority Data
May 12, 2023 (IT) 102023000009555

(51) Int. Cl. A61M 16/14; A61M 16/08; A61M 16/20; B05B 1/26; F16L 15/00

(52) U.S. Cl. CPC A61M 16/14; A61M 16/0816; A61M 16/201; B05B 1/26; F16L 15/006

(57) ABSTRACT

A device for dispensing a liquid or semi-solid product that includes an actuator button configured to couple to a dispensing valve of a container and having a first connector, and a dispensing accessory having a second connector and a dispensing outlet. The connectors are removably couplable to connect the accessory to the actuator button such that, when coupled, pressing the actuator button opens the dispensing valve to discharge the product through the dispensing outlet. The accessory cab be a variable-jet nebulizer including a hollow chamber and a shutter movable along a longitudinal axis to vary spray characteristics.

### FIELD OF THE INVENTION

The present invention relates to a device for dispensing liquid or semi-solid products, particularly medicines, medical devices or cosmetic products, as well as a kit for dispensing such products. More particularly, the present invention relates to a device for dispensing liquid or semi-solid products, in aerosol, cream, gel or liquid form. More particularly, the present invention relates to a dispensing device which can be combined with different types of containers which can be: pressurised, airless technology, Bag-On-Valve technology, aerosol and in general for all those containers having a dispensing valve for coupling to a dispensing device.

### STATE OF THE ART

As is well known, there are currently various forms of administering for liquid and semi-solid products. Different forms of administering require the use of different dispensing devices.

Moreover, even the same liquid or semi-solid product can be administered in different forms, or in any case with different dispensing devices, according to the type of application. One example is that of isotonic seawater sprays for daily nose and ear hygiene, which are often supplied with two different dispensing devices, one for cleaning the nose, the other for cleaning the ears, which can be replaced directly by the end user.

The need to provide different dispensing devices according to both the type of product to be administered and the desired mode of administering has the disadvantage of having to dedicate both special lines for production of the different dispensing devices and different lines for assembly of the dispensing devices with their respective containers, with obvious complications in the logistics of packaging and distribution of the finished product.

### SUMMARY OF THE INVENTION

In light of the above, it is a task of the present invention to make a dispensing device for liquid or semi-solid products, particularly for medicines, medical devices or cosmetic products, which overcomes the limits of the prior art by enabling standardisation of production of the dispensing devices themselves as well as the packaging of the liquid or semi-solid products contained in the containers to which such dispensing devices are attached.

Within the context of this task, it is an object of the present invention to realise a dispensing device that can be easily adapted to the specific type of dispensing to be performed.

A further object of the invention consists of the fact of realising a dispensing device that is capable of giving the broadest guarantees of reliability and safety in use.

A still further object of the invention consists of the fact of realising a dispensing device that is easy to make and economically competitive if compared to the prior art.

The above-mentioned task, as well as the aforementioned objects and others that will appear in greater detail here below, are achieved by a device for dispensing liquid or semi-solid products, particularly medicines, medical devices or cosmetic products, according to claim 1, as well as by a kit for dispensing liquid or semi-solid products according to claim 10.

Other features are provided in the dependent claims.

### LIST OF DRAWINGS

Further features and advantages will be made clearer by the description given as a non-limiting example of a preferred embodiment of the present invention, illustrated with the aid of the accompanying drawings in which:

FIG. 1 shows a kit for dispensing liquid or semi-solid products, according to the invention, comprising an actuator button which can be associated with three different dispensing accessories to obtain three different types of devices for dispensing liquid or semi-solid products;

FIG. 2 is a front elevation view of an actuator button of a dispensing device according to the invention;

FIG. 3 is a front elevation view of a dispensing accessory of a dispensing device according to the invention;

FIG. 4 is a cross section view of the actuator button shown in FIG. 2, taken along axis IV-IV;

FIG. 5 is a section view of the dispensing accessory shown in FIG. 3, taken along axis V-V;

FIG. 6 is an enlargement of the portion of FIG. 5 denoted by VI;

FIG. 7 is an enlargement of the portion of FIG. 5 denoted by VII;

FIG. 7a is an enlargement corresponding to that of FIG. 7, but referred to a different cross section, and in a different configuration of use of the dispenser attachment of FIG. 3;

FIG. 8 is a plan view from above of the dispensing accessory of FIG. 3;

FIG. 9 is an enlargement of the portion of FIG. 8 denoted by IX;

FIG. 10 is a section view of a different embodiment of a device for dispensing liquid or semi-solid products, according to the invention.

### DETAILED DESCRIPTION OF THE INVENTION

With particular reference to the drawings, the device for dispensing liquid or semi-solid products, particularly medicines, medical devices or cosmetic products is denoted overall by reference numeral 1.

According to the invention, the dispensing device 1 comprises:
an actuator button 2, configured to be coupled to a dispensing valve of a container of a liquid or semi-solid product, where this actuator button 2 comprises a first connector 3;
a dispensing accessory 10, 20, 30 comprising a second connector 4 and a dispensing mouth 5.

The first connector 3 and the second connector 4 are removably coupled one to the other to connect, in a coupling configuration, the dispensing accessory 10, 20, 30 to the actuator button 2. In the coupling configuration, pressing of the actuator button 2 is apt to activate the dispensing valve of the container of the liquid or semi-solid product to dispense a quantity of this product from the dispensing mouth 5 of the dispensing accessory 10, 20, 30.

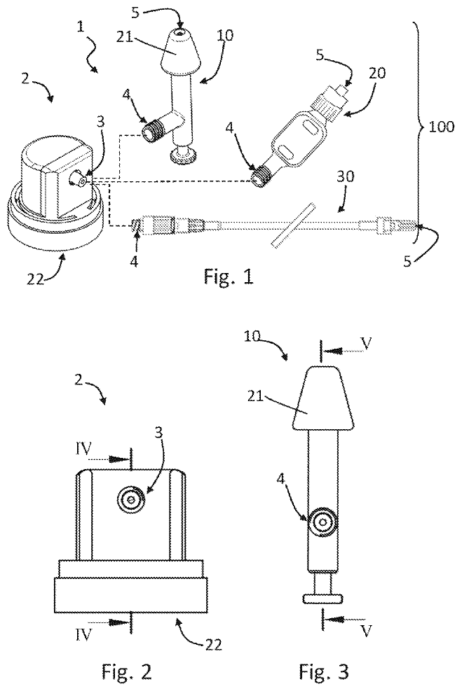

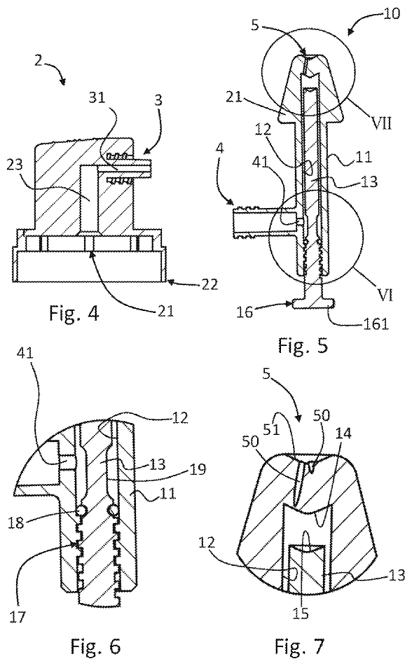

As illustrated in particular in FIG. 1, the present invention also relates to a kit 100 for dispensing liquid or semi-solid products, particularly medicines, medical devices or cosmetic products, comprising:
an actuator button 2, configured to be coupled to a dispensing valve of a container of a liquid or semi-solid product, where this actuator button 2 comprises a first connector 3;
a dispensing accessory 10, 20, 30, wherein each dispensing accessory 10, 20, 30 comprises a second connector 4 and a dispensing mouth 5.

The first connector 3 and the second connector 4 can be removably coupled one to the other to connect, in a coupling configuration, a dispensing accessory 10, 20, 30, selected from said plurality of dispensing accessories 10, 20, 30, to the actuator button 2. In the coupling configuration, pressing of the actuator button 2 is apt to activate the dispensing valve of the container of the liquid or semi-solid product to dispense a quantity of this product from the dispensing mouth 5 of the dispensing accessory 10, 20, 30.

FIG. 1 illustrates, purely by way of example, three different types of dispensing accessories 10, 20, 30 which can be used with a same actuator button 2. These are a nebuliser dispensing accessory 10, which will be described in more detail below, a dispensing accessory with filter function 20 and a dispensing accessory with catheter function 30.

Advantageously, the dispensing device 1 and the dispensing kit 100 according to the invention make it possible to overcome the limitations due to the existence of various types of administering of liquid or semi-solid products, in particular in the case of medicines, medical devices or cosmetic products, obviating the need to have dispensing devices that are completely different one from the other. In fact, according to the invention, a same actuator button 2 can be coupled, on each occasion, to a different dispensing accessory 10, 20, 30 by means of the coupling conferred by the presence of the first connector 3 on the actuator button 2 and the second connector 4 on the dispensing accessory 10, 20, 30.

According to the type of liquid or semi-solid product contained in the container, the end user can be provided with a dispensing device 1 in which the actuator button 2 is packaged together with a single specific dispensing accessory 10, 20, 30, optionally in an already assembled configuration, or a dispensing kit 100 in which a single actuator button 2 is packaged together with a plurality of different dispensing accessories 10, 20, 30 that can be assembled as needed by the end user.

In any case, the possibility of associating different dispensing accessories 10, 20, 30 with a same actuator button 2 allows standardising both of the production phase and the phase of packaging of liquid or semi-solid products, prior to large-scale distribution.

The first connector 3 preferably comprises a female (or male) attachment and the second connector 4 comprises a male (or female) attachment complementary to the attachment of the first connector 3.

The male and female connections are preferably threaded, screw, attachments.

The first connector 3 and the second connector 4 preferably define a screw-threaded attachment of the type known by the name “luer lock”.

The first connector 3 is preferably traversed by a first channel 31 and the second connector 4 is traversed by a second channel 41. In the coupling configuration of the two connectors 3 and 4, the two channels 31 and 41 face each other in such a way as to define a channel for passage of the liquid or semi-solid product. Preferably, the two connectors 3 and 4 are reciprocally coupled in such a way as to also ensure a fluid seal on either side of them.

The actuator button 2 preferably comprises an engagement portion 21 configured to engage with the liquid or semi-solid product container dispensing valve. This engagement portion 21 is configured in such a way that pressing of the actuator button 2 towards the container actuates the dispensing valve and the latter dispenses the contents of the container within the actuator button 2 until reaching the first channel 31. The actuator button 2 also preferably comprises a lower annular portion 22 configured to hook stably to the top of the body of the container. Between the engagement portion 21 and the first channel 31 that traverses the first connector 3 there can be a connecting channel 23, such that the product dispensed from the dispensing valve first traverses the connecting channel 23 and then the first channel 31, before reaching the second channel 41 present in the second connector 4 forming part of the dispensing accessory 10, 20, 30.

The connecting channel 23, as well as the first channel 31 and the second channel 41, preferably have an inner diameter appropriately dimensioned on the basis of the product to be dispensed.

The dispensing device 1 preferably comprises a dispensing accessory which is a variable-jet nebuliser dispensing accessory 10.

The nebuliser dispensing accessory 10 preferably comprises a body 11 that develops in a preferred development direction. A hollow chamber 12, also having a preferred development direction parallel to the preferred development direction of the body 11, is formed within the body 11. The hollow chamber 12 is suitable for filling with the liquid product contained inside the container and dispensed through the respective dispensing valve.

The nebuliser dispensing accessory 10 further comprises a shutter 13 movable inside the hollow chamber 12, in a direction parallel to the preferred development direction of the hollow chamber 12 itself. The speed of ejection of the liquid product from the dispensing mouth 5 of the nebuliser dispensing accessory 10 varies as a function of the position of the shutter 13 inside the hollow chamber 12.

The dispensing mouth 5 of the nebuliser dispensing accessory 10 preferably comprises a plurality of holes 50 which fluidly connect the external environment with the internal volume of the hollow chamber 12. The shutter 13 is movable from a position of maximum distance from said holes 50 to a position of at least partial obstruction of said holes 50.

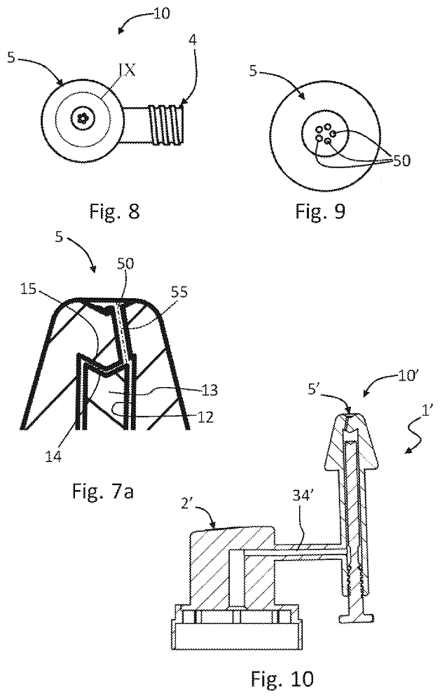

The holes 50 preferably face a bottom wall 14 of the hollow chamber 12. As illustrated in particular in FIG. 7 and in FIG. 7a, this bottom wall 14 has a substantially conical shape. The shutter 13 has an axial end 15, turned towards the bottom wall 14, which has a substantially complementary shape to the substantially conical shape of the bottom wall 14 of the hollow chamber 12.

The axial end 15 of the shutter 13 preferably has a recess with a conical shape, while the bottom wall 14 of the hollow chamber has a projection with a conical shape substantially complementary to the recess with conical shape.

This shape coupling between the bottom wall 14 of the hollow chamber 12 and the axial end 15 of the shutter 13 makes obstruction of the holes 50 more effective when the shutter 13 is pushed in proximity to and/or against the bottom wall 14 of the hollow chamber 12.

The shutter 13 preferably comprises a command portion 16 protruding externally with respect to the body 11 and suitable for being manipulated by a user to move the shutter 13 inside the hollow chamber 12.

The shutter 13 is preferably associated with the body 11 of the nebuliser dispensing accessory 10 by means of a screw thread 17, where rotation of the command portion 16 entails a translation of the shutter 13 along the hollow chamber 12.

The shutter 13 preferably has a male thread coupled to a complementary female thread formed on the inner lateral surface of the hollow chamber 12.

The command portion 16 of the shutter 13 can for example comprise a wheel 161 integral with the shutter 13 itself, which can be rotated to move the shutter 13 along the hollow chamber 12. Preferably, as illustrated in FIG. 9, the holes 50 of the dispensing mouth 5 are five in number.

The holes 50 preferably develop in a direction of development incident to the preferred development direction of the body 11 of the nebuliser dispensing accessory 10. More particularly, the direction of development of the holes has an angle of inclination with the preferred development direction of the body 11 comprised between 10 ° and 30 °, preferably approximately 20°.

In this way, the jets of liquid exiting each hole 50, in the direction of development of the holes 50 themselves, converge and collide one with the other, at a distance comprised between 2 and 4 millimetres from the upper face 51 of the dispensing mouth 5, preferably at a distance of 3 mm, nebulising themselves.

As illustrated in particular in FIG. 7a, the diameter of the shutter 13 is dimensioned in such a way that, once the position of at least partial obstruction of the holes 50 has been reached, i.e. the position of abutment between the axial end 15 of the shutter 13 and the bottom wall 14 of the hollow chamber 12, the holes 50 themselves are obstructed by the axial end 15 of the shutter 13 for a coverage value of between 50% and 75% of the opening of the holes 50 themselves, preferably for a coverage value of about ⅔ of the opening of the holes 50 themselves.

As mentioned above, the holes 50 are preferably positioned in such a way that their centre distance 55 is inclined by approximately 20 ° with respect to the preferred development direction of the body 11. This inclination is such that, for each hole 50, the points of intersection between the centre distances 55 of the holes 50 and the planes on which lie the apertures of the holes 50 that face the bottom wall 14 of the hollow chamber 12, are distributed along a circumference having a diameter approximately equal to or slightly smaller than the outer diameter of the shutter 13 (for example, smaller by a value comprised between 3% and 15%). In this way, when the axial end 15 of the shutter 13 abuts the bottom wall 14 of the hollow chamber 12, the axial end 5 of the shutter 13 couples to the bottom wall 14 of the hollow chamber 12 obstructing the holes 50 according to the coverage value described above.

A liquid sealing system is preferably provided between the shutter 13 and the side walls of the hollow chamber 12, which can for example comprise an elastomeric sealing ring 18. As illustrated in particular in FIG. 6, this elastomeric sealing ring 18 can be placed between the second channel 41 of the second connector 4 and the screw thread 17 present on the shutter 13 itself.

Alternatively, the same material in which the portion of the shutter 13 which couples, by means of threading, with the hollow chamber 12, can be such as to ensure liquid tightness.

The shutter 13 preferably has, at the portion facing the second channel 41 of the second connector 4, an annular groove 19 suitable for allowing the correct entry of pressurised liquid from the container to the hollow chamber 12, defining, substantially, a sort of storage tank of pressurised liquid.

The nebuliser dispensing accessory 10 can preferably comprise, at the top, an adapter 21 which can be made in different materials and shaped and dimensioned appropriately according to the type of administering to be performed. This adapter 21 is preferably also interchangeable, precisely so that it can be used for administering solutions in different cavities of the human body. The present invention also relates to a dispensing device l′ of liquid or semi-solid products, particularly of medicines, medical devices or cosmetic products, as shown in FIG. 10, and comprising an actuator button 2′ configured to be coupled to a dispensing valve of a container of a liquid or semi-solid product and a variable-jet nebuliser dispensing accessory 10′ comprising a dispensing mouth 5′, where the pressing of the actuator button 2′ is designed to activate the dispensing valve of the container to dispense a quantity of liquid or semi-solid product from the dispensing mouth 5′ of the nebuliser dispensing accessory 10′. With respect to the embodiment of the dispensing device 1 described above, in the dispensing device 1′ the actuator button 2′ and the nebuliser dispensing accessory 10′ are made immovably joined together, i.e. without the first and second connectors 3 and 4 for removable coupling. The technical features described with reference to the nebuliser dispensing accessory 10 illustrated in FIGS. 3 and 5-9, are also all present in the nebuliser dispensing accessory 10′ illustrated in FIG. 10, with the sole difference that the actuator button 2′ and the nebuliser dispensing accessory 10′ are in fluid communication via a channel 34′, equivalent, in function, to the union of the two channels 31 and 41 respectively of the first and second connectors 3, 4. For the description of these technical features reference is therefore made to the preceding description part.

The functioning of the device for dispensing liquid or semi-solid products, particularly medicines, medical devices or cosmetic products, as well as the relative kit, is clear and evident from what is described.

In particular, actuator button 2 acts as a multi-purpose dispensing button that can be combined, on the one hand, with different types of containers, which can be pressurised, airless technology, Bag-on-Valve technology, aerosols and in general for all those containers that have a dispensing valve, and on the other hand with a plurality of different types of dispensing accessories, capable of dispensing different liquid or semi-solid products, and of ensuring different forms of dispensing.

In fact, in addition to expanding the possibilities of use of different products, lowering the limits dictated by having to create a complex dispenser button designed to be able to administer the desired product with a single object, it is possible to offer increasingly specific and sophisticated accessories, regardless of the coupling with the container, thanks to the use of an actuator button suitable for all the different types of accessories and products.

The decision to provide a luer-lock type connection between actuator button 2 and dispensing accessory 10, 20, 30 allows virtually any type of dispensing accessory 10, 20, 30 to be combined with a single, universal actuator button 2.

The functioning of the peculiar nebuliser dispensing accessory 10 is also evident from what is described. More particularly, this accessory 10 allows dispensing devices 1 and 1′ to be obtained, capable of generating different types of nebulised jet, simply by manually actuating a shutter.

The dispensing device 1, 1′ provided with the nebuliser dispensing accessory 10 can be used with different aqueous solutions contained in different types of containers, as long as they are provided with a dispensing valve compatible with the actuator button 2. Once the dispensing valve has been actuated, pressing the actuator button 2, the pressurised liquid inside the container passes through the first and second connectors 3 and 4 and enters the hollow chamber 12. Here the pressurised liquid tries to occupy the entire available volume. After having invaded the annular groove 19, due to the presence of the watertight seal conferred by the sealing system defined, for example, by the elastomeric ring 18, the liquid fills the space of the hollow chamber 12 present between the bottom wall 14 of the same and the axial end 15 of the shutter 13 thus reaching the holes 50. At this point the liquid can exit from the holes 50 and, on the basis of the position of the shutter 13 along the hollow chamber 12, a desired and different nebulisation of the same can take place.

More particularly, in the case of liquid solution with a density comparable to that of water (about 1000 Kg/m³ ) and an operating pressure of about 7 MPa, and with five holes 50 having a diameter of 0.3 mm each (when not obstructed), the exit velocity of the jet of aqueous solution is approximately 5 m/s when the shutter 13 is at the maximum distance from the holes 50, and approximately 14 m/s when the shutter 13 is at such a distance from the holes 50 that they are partially obstructed (specifically, they have a gap of equivalent diameter of approximately 0.1 mm). As the speed at which the five jets of aqueous solution collide increases, finer nebulisation takes place. Broadly speaking, with the shutter 13 in the high position, i.e. against the bottom wall 14 of the hollow chamber 12, the particles of aqueous solution can be between 10 and 20 microns in size, while with the shutter 13 in the low position, the particles of aqueous solution maintain a larger size of about 50 microns.

The same functioning also occurs in the case of the dispensing device 1′ in which the actuator button 2 and the nebuliser dispensing accessory 20 are integrated one with the other.

In practice, it has been found that the device and kit for dispensing liquid or semi-solid products, particularly medicines, medical devices or cosmetic products, according to the present invention, fulfils the task as well as the objects set in that it allows the use of a same actuator button, substantially universal, to dispense different types of liquid or semi-solid products with different types of dispensing.

Another advantage of the device, according to the invention, consists of the fact of making the production of dispensing devices and the packaging of liquid or semi-solid products contained in containers to which such dispensing devices are applied simpler and more standardised, and therefore more economically competitive.

A further advantage of the device, according to the invention, consists of the fact that it can easily be adapted, both during industrial assembly and by the end user, to the specific type of administering to be carried out.

Yet another advantage of the dispensing device, according to the invention, consists in the fact that the end user can vary as required the features of the nebulised jet dispensed.

The device and kit for dispensing liquid or semi-solid products, particularly medicines, medical devices or cosmetic products thus designed can undergo numerous modifications and variations all within the scope of the inventive concept.

Moreover, all details can be replaced by other technically equivalent elements.

In practice, the materials used, provided they are compatible with the specific use, as well as the contingent dimensions and shapes may be any type, according to needs.

1. A device for dispensing liquid or semi-solid products comprising:
an actuator button configured to be coupled to a dispensing valve of a container of a liquid or semi-solid product, and comprising a first connector; and
a dispensing accessory comprising a second connector and a dispensing mouth;
wherein said first connector and said second connector are configured to be removably coupled one to the other to connect, in a coupling configuration, said dispensing accessory to said actuator button, and
wherein, in said coupling configuration, pressing of said actuator button is configured to activate said dispensing valve of said container to dispense a quantity of said liquid or semi-solid product from said dispensing mouth of said dispensing accessory.

2. The device according to the claim 1, wherein said first connector comprises a female attachment and said second connector comprises a male attachment, complementary to said female attachment of said first connector.

3. The device, according to the preceding claim, wherein said first connector and said second connector form a luer-lock threaded connection.

4. The device according to claim 1, wherein said dispensing accessory is a variable-jet nebuliser dispensing accessory.

5. The device, according to claim 4, wherein said nebuliser dispensing accessory comprises a body defining a longitudinal axis; hollow chamber with the body extending along the longitudinal axis and configured to receive the product from the container; and a shutter movable within said hollow chamber along the longitudinal axis, wherein the speed of ejection of said liquid product from said dispensing mouth varying as a function of the position of said shutter inside said hollow chamber.

6. The device according to claim 5, wherein said dispensing mouth comprises a plurality of holes that place the external environment in fluid communication with the internal volume of said hollow chamber, said shutter being movable from a position of maximum distance from said holes to a position of at least partial obstruction of said holes.

7. The device according to claim 6, wherein said holes face a bottom wall of said hollow chamber, said bottom wall having a substantially conical shape, said shutter comprising an axial end that has a shape substantially complementary to said substantially conical shape of said bottom wall of said hollow chamber.

8. The device according to claim 5 wherein said shutter comprises a command portion protruding externally with respect to said body and configured for manual adjustment by a user to move said shutter inside said hollow chamber.

9. The device, according to claim 8, wherein said shutter is associated with said body by means of a screw thread, wherein rotation of said command portion causes translation of said shutter along said hollow chamber.

10. A dispensing kit for liquid or semi-solid products comprising
an actuator button configured to be coupled to a dispensing valve of a container of a liquid or semi-solid product, and comprising a first connector; and
a plurality of dispensing accessories, wherein each dispensing accessory comprises a second connector and a dispensing mouth;
wherein said first connector and said second connector can be removably coupled one to the other to connect, in a coupling configuration, a dispensing accessory selected from said plurality of dispensing accessories to said actuator button, and
wherein, in said coupling configuration, the pressing of said actuator button opens the dispensing valve to discharge the product through the dispensing outlet.

11. A dispensing device of liquid or semi-solid products comprising an actuator button configured to be coupled to a dispensing valve of a container of a liquid or semi-solid product and a variable-jet nebuliser dispensing accessory comprising a dispensing mouth wherein the pressing of said actuator button opens the dispensing valve to discharge the product through the dispensing outlet.
